(19) United States
(12) Patent Application Publication
(10) Pub. No.: US 2020/0100331 A1
(43) Pub. Date: Mar. 26, 2020

(54) Portable Foot Warmer

(71) Applicant: Meng Penguin, San Mateo, CA (US)

(72) Inventor: Meng Penguin, San Mateo, CA (US)

(21) Appl. No.: 16/136,672

(22) Filed: Sep. 20, 2018

(51) Int. Cl. H05B 3/34; A61F 7/00

(52) U.S. Cl. CPC H05B 3/342; A61F 2007/0045; H05B 2203/003; A61F 7/007

(57) ABSTRACT

A portable foot warmer with one or more heating pads, a signal receiving circuit, a signal transmitting circuit, a transmitting circuit housing, an AC to DC power supply, a receiver and power supply housing and one or more electrical cables connecting the AC to DC power supply to the heating pads. The heating pads are thin and flexible. The signal transmitting circuit is capable of transmitting to the receiving circuit to turn on or off and select heat level of the heating pad. A preferred embodiment includes the heating pad is flexible, approximately twelve inches wide, twenty-four inches long and approximately fifty thousandths of an inch thick.

### CROSS REFERENCE TO RELATED APPLICATION

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of portable heating devices and more specifically to a portable foot warmer for use in bed.

During colder months of the year, a person's feet may experience an unwanted cold effect while sleeping in bed.

To remedy this condition, various solutions include the use electric warming blankets, hot water bottles and heavy socks.

However, electric blankets tend to heat the entire body, which is not ideal of it is only desired to keep the feet warm. Hot water bottles eventually cool down and socks tend to be uncomfortable and restricting. Therefore, it would be ideal to have a heating source for warming the feet while in bed that can be easily controlled, easily installed and can be selected for one or two people sleeping in the bed.

### BRIEF SUMMARY OF THE INVENTION

The primary object of the invention is to provide a portable foot warmer that can easily be attached to a mattress or bed sheet and can warm the user's feet on cold nights.

Another object of the invention is to provide a portable foot warmer that can be turned on or off remotely.

Another object of the invention is to provide a portable foot warmer that can be a single warmer for a single occupant of a bed, or can be a double warmer that can be used by two occupants of a bed.

A further object of the invention is to provide a portable foot warmer that allows the power cable to be hidden between the mattress and box spring portion of the bed.

Yet another object of the invention is to provide a portable foot warmer that is very flexible, soft and thin in profile so that it does not interfere with normal sleeping activities.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a portable foot warmer comprising: one or more heating pads, a signal receiving circuit, a signal transmitting circuit, a transmitting circuit housing, an AC to DC power supply, a receiver and power supply housing, one or more electrical cables connecting said AC to DC power supply to said heating pads, said heating pads being thin and flexible and said signal transmitting circuit capable of transmitting to said receiving circuit to turn on and select heat level of said heating pad.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a perspective view of the invention.

FIG. 2 is a perspective view of the invention with the power cables hidden between the mattress and box spring.

FIG. 3 is a perspective view of a single heating pad configuration.

FIG. 4 is a perspective view of a single heating pad configuration in use with the power cord hidden.

FIG. 5 is a perspective view of a single heating pad showing Velcro attachment strips.

FIG. 6 is a perspective view of a double transmitter assembly.

FIG. 7 is a perspective view of a single transmitter assembly.

FIG. 8 is an exploded view showing the transmitter assembly ready to be inserted into a cradle.

### DETAILED DESCRIPTION OF THE DRAWINGS AND PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

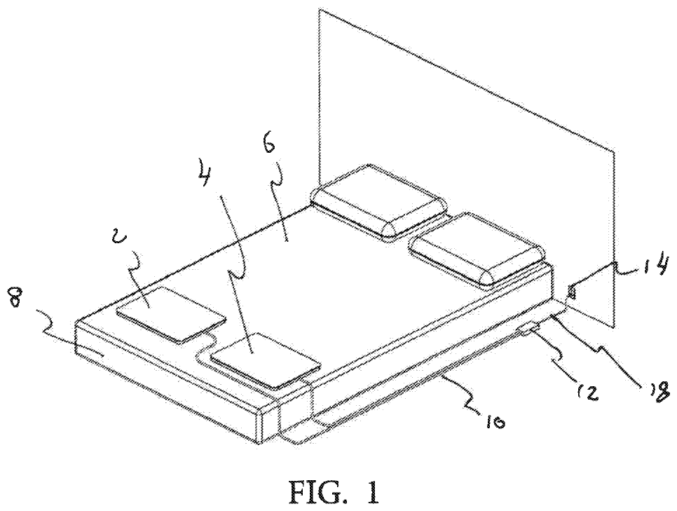

Referring now to FIG. 1 we see a perspective view of main components of the invention consisting of heating pads 2, 4, electric cables 10, 14 and power supply housing 12. The heating pads 2, 4 are located at the foot of a mattress 8. The pads can be held in place by hook and loop fastening strips or other standard means. The pads 2, 4 can be affixed to the top surface of a mattress 8 or to the top surface of a bed sheet 6. The heating pads 2,4 are connected by cables 10 to AC to DC power control box 12 and then, via cable 18 to a standard AC wall socket 14.

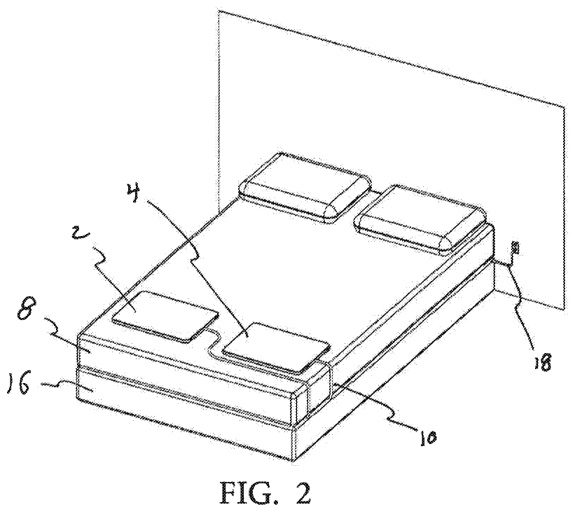

FIG. 2 is a perspective view showing the addition of a standard box spring member 16 allowing cables 10 to be tucked into the space between the mattress 8 and the box spring 16 and exiting at cable 18 Where it can plug into wall socket 14.

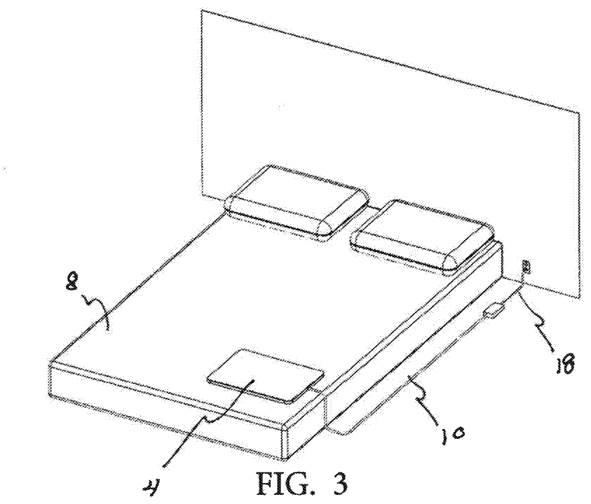

FIG. 3 is a perspective view of a single heating pad 4 being used. This format is ideal for single person beds or for a couple where only one person needs the advantage of a foot warmer.

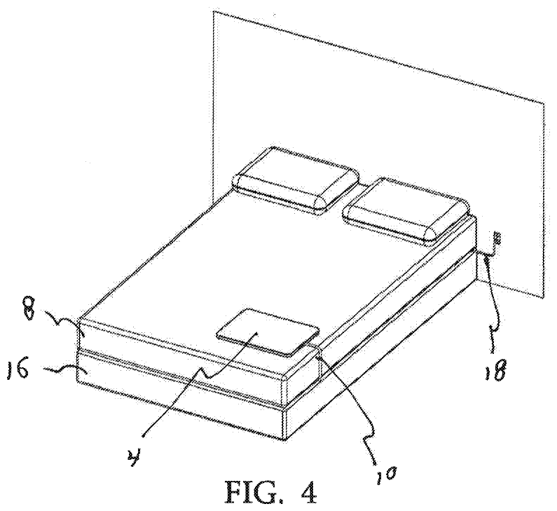

FIG. 4 is a perspective view of the single heating pad version where the cables 10 are tucked into the space between the mattress 8 and the box spring 16.

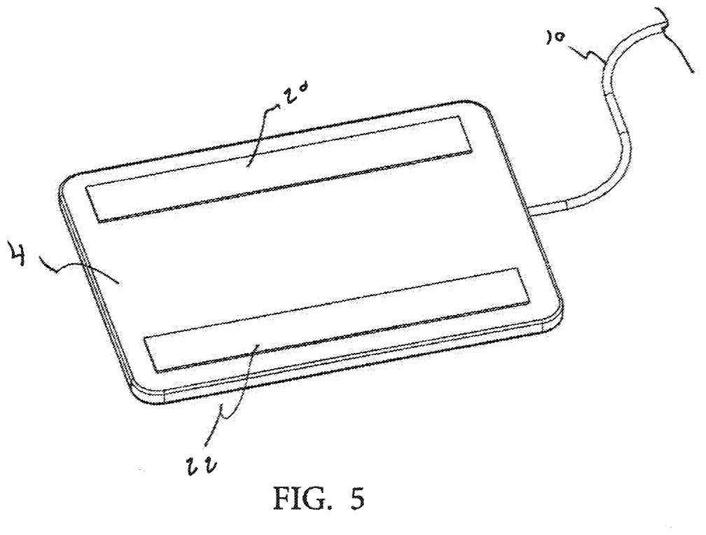

FIG. 5 is a perspective view of a heating pad 4 showing the underside having Velcro or hook and loop fastening strips 20, 22 that are attached to the heating pad 4 on one side and mating strips on a mattress top or sheet top. The heating pads are extremely thin and flexible, and include a felt-like top surface, so they can be attached to a bed sheet and not be a physical hindrance to normal sleeping habits. The pad 4 includes a top layer, a bottom layer and a central layer that has a printed carbon heating element screen printed in place. The overall size of the heating pad 4 is approximately twelve inches wide, twenty-four inches long and fifty thousandths of an inch thick.

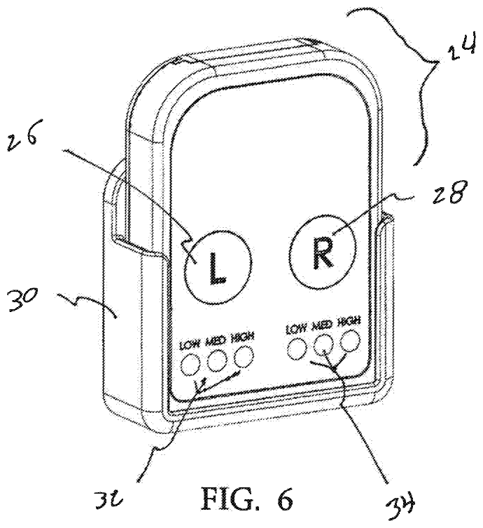

FIG. 6 is a perspective view of the two-pad transmitter 24 housed in a holding cradle 30. The housing 24 holds a transmitting circuit, LED indicator lights, an on off switch and a battery power supply. The user can press either the switch labeled “L” or “R” or both which can activate the left heating pad 2 or the right heating pad 4. By pressing either switch multiple times, the user can select a low, medium or high setting which is indicated by indicating lights 32, 34. The transmitting circuit can send a signal to a receiving circuit which is housed within the AD to DC power supply box 12. The power supply is plugged into an AC wall socket 14 via cable 18.

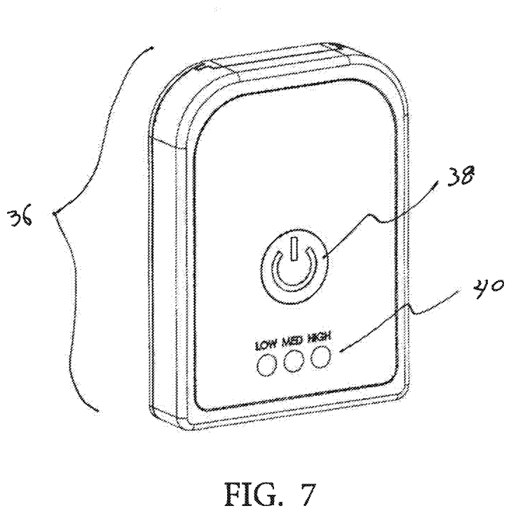

FIG. 7 is a perspective view of a single transmitter housing 36 having a single activation switch 38 which can be pressed multiple times to set the low, medium or high setting as shown by indicator lights 40.

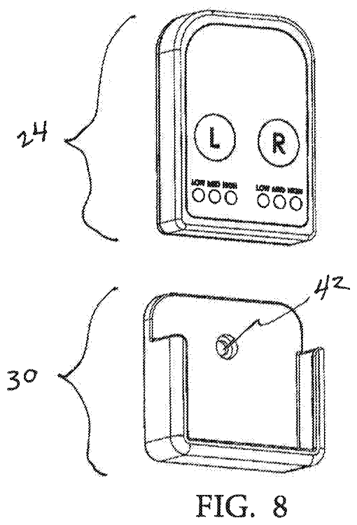

FIG. 8 is an exploded view showing how the transmitter housing 24 can be removed or replaced from cradle 30 which can be attached to a wall or other flat surface via a screw that can be inserted into aperture

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. Portable foot warmer comprising:
one or more heating pads;
a signal receiving circuit;
a signal transmitting circuit;
a transmitting circuit housing;
an AC to DC power supply;
a receiver and power supply housing;
one or more electrical cables connecting said AC to DC power supply to said heating pads;
said heating pads being thin and flexible and
said signal transmitting circuit capable of transmitting to said receiving circuit to turn on and select heat level of said heating pad;

2. Portable foot warmer as claimed in claim 1 wherein said heating pad is flexible, approximately twelve inches wide, twenty-four inches long and approximately fifty thousandths of an inch thick.

3. Portable foot warmer as claimed in claim 1 wherein said heating pad is constructed of an upper fabric layer and a lower fabric layer and a central flexible layer having a screen-printed heating element.

4. Portable foot warmer as claimed in claim 1 wherein said signal transmitting circuit can transmit instructions to one of two heating pads enabling two people to each choose their own level of heat.

5. Portable foot warmer as claimed in claim 1 wherein said electrical cables can be hidden between a mattress and box spring portion of a standard bed.

6. Portable foot warmer as claimed in claim 1 wherein said heating pads can be attached to the top portion of a mattress, or the top portion of a sheet by means of hook and loop fastening strips or other standard fastening means.
